(19) United States
(12) Patent Application Publication
(10) Pub. No.: US 2016/0158064 A1
(43) Pub. Date: Jun. 9, 2016

(54) MASK WITH A COMFORT ELEMENT

(71) Applicant: SALOMON S.A.S., Metz-Tessy (FR)

(72) Inventors: Herve FAVRE-FELIX, Villaz (FR); Thierry DONNADIEU, Sillingy (FR)

(73) Assignee: SALOMON S.A.S., Metz-Tessy (FR)

(21) Appl. No.: 14/959,481

(22) Filed: Dec. 4, 2015

(30) Foreign Application Priority Data
Dec. 5, 2014 (FR) 14/02790

(51) Int. Cl. A61F 9/02

(52) U.S. Cl. CPC A61F 9/026

(57) ABSTRACT

An eye protective mask designed for the practice of outdoor sports, for example, includes an eye protective screen, a frame supporting the screen, and a comfort element affixed to the frame. The comfort element includes a nasal portion configured to come into contact with a portion of the user's nose, and a frontal portion configured to come into contact with a portion of the user's forehead. The material used for the nasal portion has different mechanical compression properties from the material used for the frontal portion, such that the maximum compression, or stroke, of the material of the nasal portion caused by a predetermined compressive force is greater than the compression of the material of the frontal portion caused by the same predetermined compressive force.

### CROSS-REFERENCE TO RELATED APPLICATION

This application is based upon French Patent Application No. FR 14/02790, filed Dec. 5, 2014, the disclosure of which is hereby incorporated by reference thereto in its entirety, and the priority of which is claimed under 35 U.S.C. §119.

### BACKGROUND

1. Field of the Invention

The invention relates to eye protective masks, particularly protective masks for the practice of outdoor sports, such as alpine skiing, snowboarding, mountain biking, motocross, and others.

2. Background Information

Protective masks, such as protective eyewear, or goggles, are typically used in the practice of certain outdoor sports to protect the user's eyes from wind, water, mud, and/or solar radiation.

A mask of the aforementioned type usually includes a frame supporting a transparent protective shield and a strap attached to opposite sides of the frame. The strap keeps the mask on the user's face by partially surrounding the user's head. Comfort elements are usually added to the frame to improve the wearing comfort of the mask. These comfort elements are positioned at the interface between the frame and the user's face. They often form a foam strip surrounding the eyes by taking support on a portion of the forehead, the temples, the cheekbones, and a portion of the nose of the user.

These comfort elements are characterized by damping and roughness properties of the contact surface. Indeed, in order to keep the mask in the area of the eyes, it is necessary to exert a force, via the strap, to maintain the frame pressed on the face. Without comfort elements, the contact with the plastic frame on the face is not pleasant. In addition, the frame does not deform much and thus does not enable good adaptation to the various morphologies of the face. The airtightness of the inner volume of the mask is not very good. Drafts can penetrate into this volume, thereby causing discomfort for the eyes. To overcome these problems, a comfort foam is usually added to the frame. Such foam enables adaptation to the morphology of the face and reduction in the contact pressure, and provides softness and the desired insulation.

The patent document U.S. 2011/0289662 proposes a comfort element made of viscous foam. This material adapts well to the morphology, within the limit of the possible local movement of the foam. Nevertheless, this material is not easy to shape.

The patent document U.S. 2013/0067626 provides a multi-layered comfort element, which is thicker in the lateral zones than in the upper and lower central zones. This construction enables the lateral zones to be properly pressed when the user is wearing a helmet.

The comfort elements of the prior art substantially have the same compression or cushioning characteristics on the eye contours. They often have a substantially constant and small thickness, at least in the contact zone of the forehead and the nose. However, the morphology of the face varies considerably from one individual to another, particularly in the area of the nose. Thus, depending upon the morphology of the user, the morphological adaptation is substantially similar in the frontal and natal zones, which is not very comfortable. The nose can then be subjected to unpleasant pressure. Moreover, the nose may be not be properly wrapped. The imperviousness of the mask may not be as good from one individual to another due to the generally small deformation of the comfort element in the nasal zone.

### SUMMARY

The present invention provides an improved mask providing better wearing comfort.

In particular, the invention provides better adaption of a mask to various morphologies of the users.

The invention also makes it possible to control the distribution of the pressure holding the mask onto the user's face in defined zones.

In addition, the invention reduces the pressure exerted in the area of the nose.

Furthermore, the invention improves the imperviousness of the inner volume of the mask.

To these ends, the invention provides an eye protective mask for the practice of outdoor sports including:
an eye protective screen;
a frame supporting the screen;
a comfort element affixed to the frame, the comfort element comprising a nasal portion configured to come into contact with a portion of the user's nose, and a frontal portion configured to come into contact with a portion of the user's forehead.

The material used for the nasal portion of the mask has mechanical compression properties that are different from the material used for the frontal portion, so that the maximum compression, or stroke, of the material of the nasal portion, caused by a predetermined compressive force thereon, is greater than the maximum compression, or stroke, of the material of the frontal portion caused by the same predetermined compressive force.

Because of this construction, the nasal portion of the comfort element of the mask can deform more than the frontal portion. Therefore, it can better conform to the complex geometry of the nose and exert less pressure in the area of the sides of the nose, which are sensitive zones. The user's breathing is not disturbed because the nostrils remain fully open, as the sides of the nose are not crushed or compressed. By more closely following the curves of the nose, the comfort element improves the imperviousness in this zone.

According to advantageous but not essential aspects of the invention, such a mask can incorporate one or more of the following features, taken in any technically permissible combination:
At least one constituent material of the nasal and/or frontal portions is a composite, the material then comprising a plurality of layers of various materials.
The material of the frontal portion and/or the material of the nasal portion successively comprises, from the frame, a first foam layer of a first type and a second foam layer of a second type, the density of the foam of the first layer being greater than the density of the foam of the second layer.
The thickness of the second layer of the nasal portion is greater than the thickness of the second layer of the frontal portion.
The two second layers are made of the same type of foam.
The density of the foam of the second layer of the nasal portion is less than the density of the foam of the second layer of the frontal portion.
The foam of the first layer of the nasal portion is of a different type than the foam of the first layer of the frontal portion.
The thickness of the nasal portion is greater than the thickness of the frontal portion.
The comfort element comprises two lateral portions, each lateral portion being adapted to come into contact with the temple and cheekbone on one side of the user's face, each lateral portion connecting one end of the frontal portion to one end of the nasal portion.
The material used for the lateral portions has intermediate mechanical compression properties compared to the materials used for the nasal and frontal portions.
The number of layers of material between the various portions of the comfort element varies.
The comfort element comprises at least two distinct elements, a first element forming the nasal portion, and the second element forming the frontal portion.
The comfort element comprises four separate elements: a frontal portion, a nasal portion, and two lateral portions.
At least a portion of the comfort element is thermoformed.
At least a portion of the comfort element is removable.

### BRIEF DESCRIPTION OF THE DRAWINGS

Other characteristics and advantages of the invention will be better understood from the description which follows, with reference to the annexed drawings illustrating, by way of non-limiting embodiments, how the invention can be carried out, and in which:

FIG. 1 is a front perspective view of the mask according to a first embodiment of the invention;

FIG. 2 is a rear perspective view of the mask without a strap;

FIG. 3 is a bottom view of the mask without a strap;

FIG. 4 is a simplified rear view of the mask without a strap;

FIG. 5 is a simplified rear view of a second embodiment; and

FIG. 6 is a simplified rear view of a third embodiment.

### DETAILED DESCRIPTION

The mask 1 comprises a frame 2 supporting an eye protective shield or lens 3. A strap 4 is attached to the lateral sides of the frame 2. The strap 4 and the frame 2 form a loop adapted to surround the user's head to maintain the mask in place.

The following description makes use of terms such as “horizontal”, “vertical”, “upper”, “lower”, “top”, “bottom”, “front”, “rear”, “anterior”, “posterior”. These terms must be considered as relative terms in relation to the normal position that the mask occupies on the face of the user, when standing with the head straight. The term “rear” refers herein to the portions oriented toward the head/eyes of the user.

The frame 2 supports a comfort element 5 arranged at the rear of the frame, at the interface between the frame and the user's face. The comfort element is configured to come into contact with the zones of the face surrounding the eyes.

The comfort element 5 comprises at least a frontal portion 51 configured to come into contact with a portion of the user's forehead, and a nasal portion 52 configured to come into contact with a portion of the user's nose. The frontal portion covers a zone of the forehead located substantially above the eyebrows of the user. The comfort element 5 thus presses on a “hard” zone due to the presence of the frontal bone of the skull. The nasal portion covers the nose bridge, the sides of the nose, at the boundary of the sides of the nose. The nasal portion can also extend over a medial portion of the cheekbones. In this region, the comfort element 5 thus presses on a “soft” and sensitive zone due to the presence of cartilage that is more fragile than the cranial bone.

The comfort element 5 can be a unitary element incorporating the frontal portion and the nasal portion. Alternatively, the comfort element 5 can comprise a plurality of distinct elements, each element being connected to the frame 2. For example, the frontal portion and nasal portion are two separate elements.

The comfort element 5 comprises at least two portions. Each portion corresponds to a specific support zone on the face. Each portion is made of a material having specific mechanical properties. Within the meaning of the invention, a material can be a single-layered material or a composite material comprised of a plurality of layers of various materials. In the latter case, this material corresponds to a “lamination”.

The material used for the nasal portion of the comfort element 5 has different mechanical compression properties from the material used for the frontal portion, so that the maximum compression, or stroke, of the material of the nasal portion material caused by a predetermined compressive force is greater than the compression, or stroke, of the frontal portion material caused by the same predetermined compressive force. In other words, if the same predetermined compressive force is exerted on the frontal portion and on the nasal portion, the nasal portion will be more greatly deformed.

The compression of a portion of the comfort element corresponds to a compression of the comfort element in its thickness, that is, along a horizontal direction translating the compression of the comfort element 5 between the user's face and the frame 2. The compressive force is therefore a force exerted in a direction X oriented along the thickness of the comfort element. The extent of compression corresponds to the variation of the position of the face on which the compressive force is exerted along the direction X. This extent is equal to the difference between the position of the face not biased and the position of the face biased by the predetermined compression force.

To obtain this difference in extent of compression, or stroke, e.g., between the front and nasal portions, several solutions are within the scope of the invention.

In a first case, at least one layer of the nasal portion 52 is made of a softer material than the material of at least one layer of the frontal portion 51. Consequently, the softer material of the nasal portion 52 can deform more than the material used for the frontal portion 51. For example, foams having various densities can be used. For the nasal portion 52, a foam having a lower density than the foam used for the frontal portion 51 can be used. Thus, by being less dense, the foam of the nasal portion deforms more than the foam of the frontal portion. The nasal and/or frontal portion can be single-layered or multi-layered. The thickness of the nasal portion can be the same as or different from the thickness of the frontal portion.

In a second case, the thickness of at least one layer of the nasal portion 52 is greater than the thickness of at least one layer of the frontal portion 51. By being thicker, the layer of the nasal portion can be deformed over a greater distance. The material used for the layer of the nasal portion 52 can be the same as or different from the material used for the layer of the frontal portion 51. For example, foams having the same density or different densities can be used. Here again, the nasal and/or frontal portion can be single-layered or multi-layered.

Thus, to modify the mechanical compression properties of a material, the type of material used for at least one layer can be changed and/or the thickness of at least one layer can be modified, whether the material is single-layered or multi-layered.

To make a layer of the nasal portion and/or the frontal portion, a polyurethane foam (PU) is advantageously used. Alternatively, the use of other materials, enabling a more substantial deformation for less force, can be considered. For certain layers, other types of foams, such as foams made of polyvinyl chloride (PVC), polyethylene (PE), ethylene vinyl acetate (EVA), can also be used.

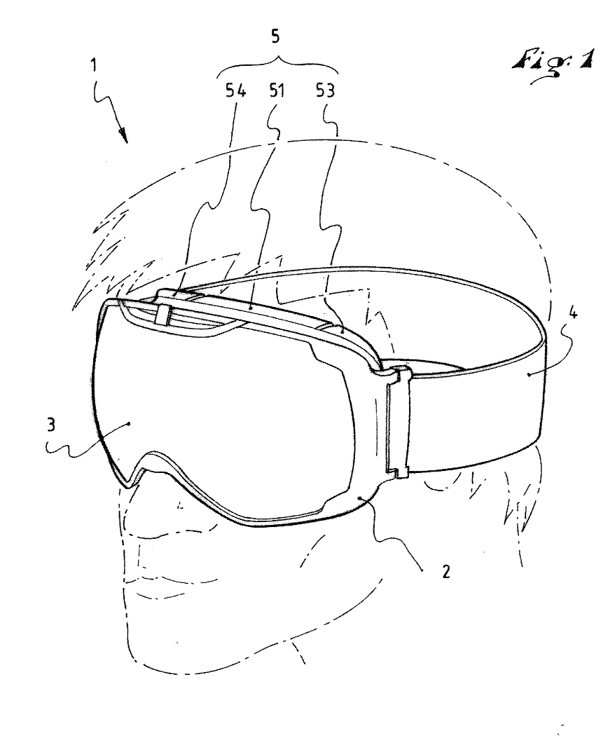

In the example illustrated in FIGS. 1 to 3, the comfort element 5 comprises a plurality of separate elements, or portions, fixed to the rear surface of the frame 2.

A first portion constitutes the frontal portion 51, having a thickness e51, covering a portion of the forehead of the user. This frontal portion is made of a laminated material, that is, it comprises a plurality of layers. A first layer 511, having a thickness e511, is fixed to the rear surface of the frame 2. In this example, this first layer 511 is made of a first type of foam: a foam made of polyvinyl chloride. A second layer 512, having a thickness e512, is fixed to the first layer 511. It follows the first layer in the thickness direction, that is, in a direction away from the frame 2. In this example, this second layer is made of a second type of foam: a polyurethane foam having a density on the order of 20 kg/m³. Finally, a thin fabric 513 is attached to the second layer 512. It follows the second layer 512 in the thickness direction. The fabric 513, or fabric layer, provides contact comfort for the user. This fabric advantageously has properties of softness on contact, breathability, and moisture absorption for the perspiration. The arrangement of the layers is such that the thickness e51 of the frontal portion corresponds to the sum of the thicknesses e511 of the first layer, e512 of the second layer, and of the fabric. The material of the frontal portion 51 successively comprises, from the frame 2, a first layer 511, a second layer 512, and a fabric 513. In one embodiment, the thickness e511 of the first layer is between 30% and 50% of the thickness e51 of the frontal portion. The thickness e512 of the second layer is between 50% and 70% of the thickness e51 of the front portion. For example, the thickness e511 of the first layer is on the order of four millimeters and the thickness e512 of the second layer is on the order of eight millimeters. In the frontal zone, the comfort element 5 must ensure good retention of the frame 2, in particular good vertical retention. For this, the pressure of contact with the user's face must be substantial enough to prevent the mask from wobbling or moving vertically. Furthermore, the presence of the frontal bone makes it possible for the user to accept a fairly significant amount of pressure without discomfort. Thus, the frontal portion of the comfort element 5 does not need to deform significantly but, on the contrary, it must enable the frame to be properly retained with suitable pressure. It is then advantageous to have a good elastic return of this frontal portion. For this, the foam used here for the first layer 511 has a modulus of elasticity significantly higher than the foam used for the second layer 512. Comfort is provided by the second layer, the retention is provided by the “responsiveness” of the first layer.

A second element, or portion, of the comfort element 5 constitutes the nasal portion 52, having a thickness e52, covering a portion of the nose of the user. This nasal portion is made of a laminated material, that is, it comprises a plurality of layers. A first layer 521, having a thickness e521, is fixed to the rear surface of the frame 2. In this example, this first layer is made of a first type of foam: a polyurethane foam having a density on the order of 60 kg/m³. A second layer 522, having a thickness e522, is fixed to the first layer 521. It follows the first layer in the thickness direction, in a direction away from the frame 2. In this example, this second layer is made of a second type of foam: a polyurethane foam having a density on the order of 20 kg/m³. Finally, a thin fabric 523, or fabric layer, is fixed to the second layer 522. It follows the second layer in the thickness direction. The fabric provides contact comfort for the user. This fabric mainly has properties of softness on contact. The arrangement of the layers is such that the thickness e52 of the nasal portion corresponds to the sum of the thicknesses e521 of the first layer, e522 of the second layer, and of the fabric. The material of the nasal portion 52 thus successively comprises, from the frame 2, a first layer 521, a second layer 522, and a fabric 523. According to one embodiment, the thickness e521 of the first layer is between 10% and 30% of the thickness e52 of the nasal portion. The thickness e522 of the second layer is between 70% and 90% of the thickness e52 of the nasal portion. For example, the thickness e521 of the first layer is on the order of four millimeters, and the thickness e522 of the second layer is on the order of twelve millimeters. In the nasal zone, the comfort element must ensure good adaptability to the morphology of the face and good comfort. For this, the nasal portion 52 must be capable of deforming substantially to assume the shape of the face with a minimum of contact pressure. Indeed, the cartilage in this area makes this portion of the face sensitive. It is therefore advantageous to reduce the pressure exerted by the mask on this zone of the face. This also makes it possible to clear the nostrils to facilitate the user's breathing. Comfort is mainly provided by the second layer. The first layer also contributes to the comfort, enables progressive damping, and provides elasticity for the retention of the mask.

According to this embodiment, the thickness e52 of the nasal portion is greater than the thickness e51 of the frontal portion. For example, the thickness e51 of the frontal portion is between 60% and 80% of the thickness e52 of the nasal portion. For this illustration, the variation in thickness is essentially achieved by the variation in thickness e512/e522 of the second layer.

In the illustrated embodiment, the comfort element 5 comprises two additional distinct elements.

A third element, or portion, constitutes the left lateral portion 53, having a thickness e53, covering the left temple and left cheekbone of the user's face. This left lateral portion 53 connects the left end of the frontal portion 51 to the left end of the nasal portion 52. This left lateral portion 53 is made of a laminated material, that is, it comprises a plurality of layers. A first layer 531, having a thickness e531, is fixed to the rear surface of the frame 2. In this example, this first layer is made of a first type of foam: a polyurethane foam having a density on the order of 60 kg/m³. A second layer 532, having a thickness e532, is fixed to the first layer 531. It follows the first layer in the thickness direction, in a direction away from the frame 2. In this example, this second layer is made of a second type of foam: a polyurethane foam having a density on the order of 20 kg/m³. Finally, a thin fabric 533 is attached to the second layer 532. It follows the second layer in the thickness direction, from the frame 2. The fabric, or fabric portion, provides contact comfort for the user. This fabric mainly has properties of softness on contact. The arrangement of the layers is such that the thickness e53 of the left lateral portion 53 corresponds to the sum of the thicknesses e531 of the first layer, e532 of the second layer, and of the fabric. The material of the left lateral portion 53 thus successively comprises, from the frame 2, a first layer 531, a second layer 532, and a fabric 533. According to one embodiment, the thickness e531 of the first layer is between 30% and 50% of the thickness e53 of the left lateral portion. The thickness e532 of the second layer is between 50% and 70% of the thickness e53 of the left lateral portion. For example, the thickness e531 of the first layer is on the order of four millimeters, and the thickness e532 of the second layer is on the order of eight millimeters.

A fourth element constitutes the right lateral portion 54, having a thickness e54, covering the right temple and right cheekbone of the user's face. The right lateral portion 54 is symmetrically identical to the left lateral portion 53. It is constructed in an analogous manner and has the same characteristics. It comprises a first layer 541, having a thickness e541, a second layer 542, having a thickness e542, and a fabric 543.

In this example, the thickness e53, e54 of the lateral portions is less than the thickness e52 of the nasal portion, and is substantially equal to the thickness e51 of the frontal portion. Advantageously, the material used for the lateral portions 53, 54 has intermediate mechanical compression properties compared to the materials used for the nasal and frontal portions. Alternatively, the material used for the lateral portions 53, 54 can have mechanical compression properties similar to the material used for one of the nasal or frontal portions.

This construction of the comfort element 5 in distinct portions makes it easy to replace damaged or worn portions. This also makes it possible to adjust the comfort of the user by increasing or decreasing the contact pressure in certain support zones on the face. Furthermore, this improves the adaptation, or configuration, of the mask to the morphology of the face. For example, depending upon the size of the user's nose, a nasal portion having a suitable thickness can be used. To facilitate this interchangeability, the distinct portions can be provided to be independently removably mounted on the frame 2. The fastening mechanisms can be hook-and-loop fasteners (such as Velcro®), snap fasteners, clip fasteners, etc.

In these examples, the comfort element 5 forms a band having a substantially constant width, between ten and twenty millimeters. The band is a “closed” loop, surrounding the eyes, which has the general shape of a horizontally elongated rectangle. The thickness of the band can vary in accordance with the embodiments described above. In its lower portion, the band forms an inverted “V” to clear the sides of the nose so as not to interfere with breathing.

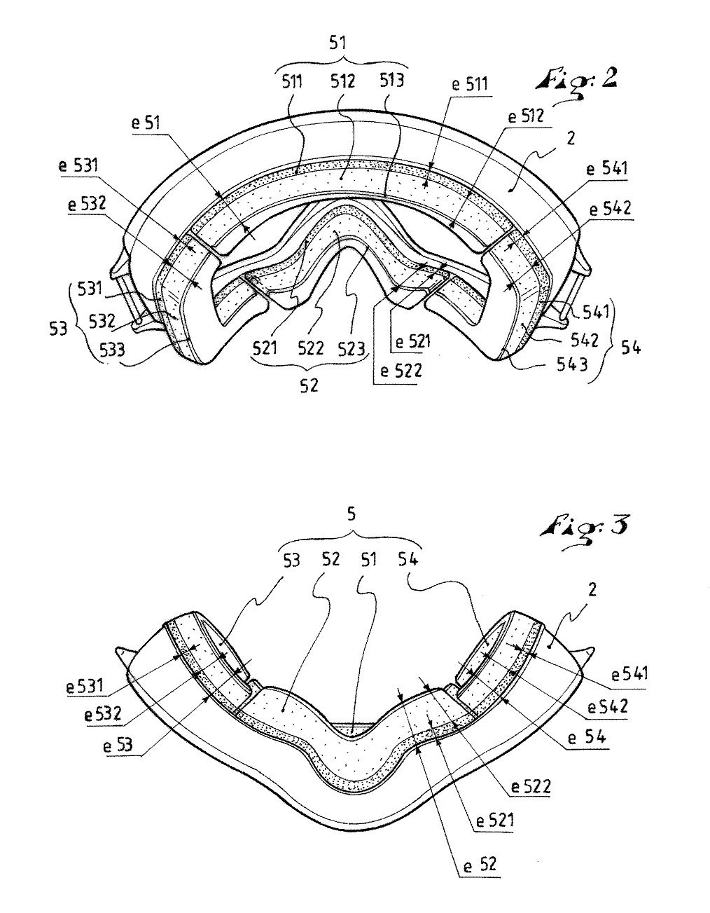

FIG. 4 is a simplified rear view of the mask shown in FIGS. 2 and 3, in the sense that the layers of the constituent portions 51, 52, 53, and 54 are not shown.

The comfort element is not necessarily comprised of four distinct elements. It can comprise more or fewer elements. For example, the comfort element can comprise only two elements.

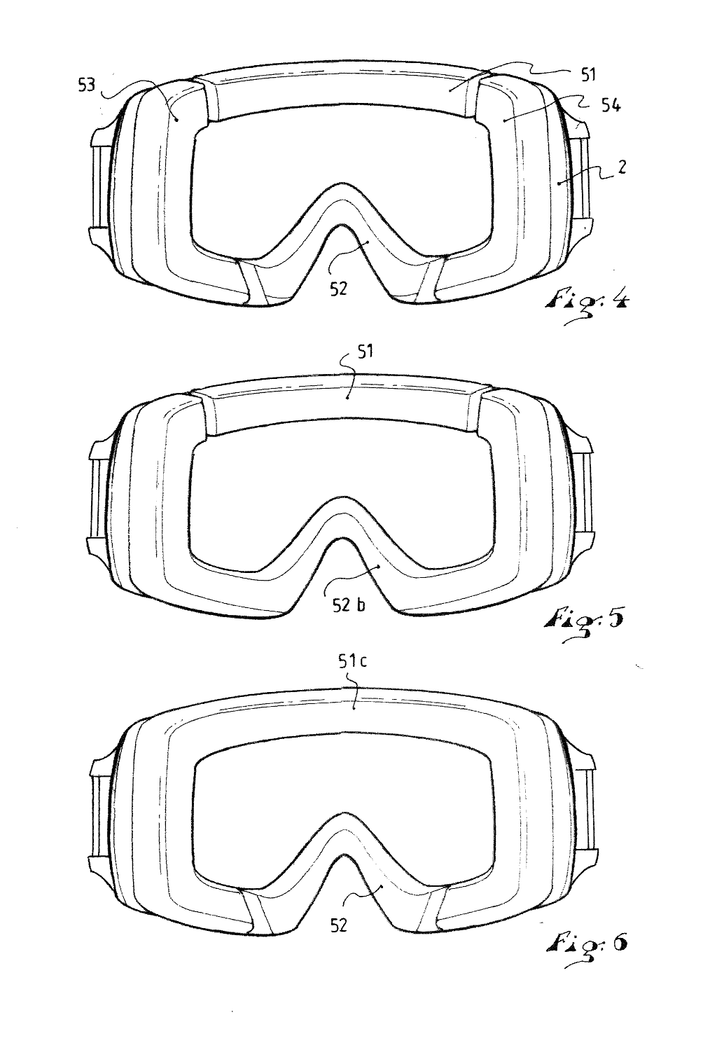

FIGS. 5 and 6 illustrate two other embodiments of a mask equipped with a comfort element comprising two elements. These drawing figures are simplified representations in the sense that the layers of the constituent portions are not shown.

FIG. 5 illustrates a second embodiment in which the comfort element comprises a front portion 51 similar to that described above, and a lower portion 52b configured to cover a portion of the nose, cheeks, and temples of the user. The lower portion corresponds substantially to the combination of the nasal portion with the two lateral portions of the first embodiment. These three distinct portions form a single continuous element. In this embodiment, the thickness of the lower portion can be variable, for example it can be thicker in the area of the nose.

FIG. 6 illustrates a third embodiment in which the comfort element includes a frontal portion 51c configured to cover a portion of the user's forehead, cheekbones and temples, and a nasal portion 52 similar to that described above. The upper portion corresponds substantially to the combination of the frontal portion with the two lateral portions of the first embodiment. These three distinct portions form a single continuous element.

Alternatively, the demarcation between the various support zones on the face can be located differently. For example, the frontal zone can cover the temples and stop in the middle of a lateral side.

In the embodiments described, the material used for the portions of the comfort element is comprised of two layers. Alternatively, the material can include more layers, for example a series of foams whose density varies gradually. This allows for a good morphological adaptability and a gradual damping/deformation, which improves the wearing comfort.

According to another embodiment, the material is a single-layered material.

Alternatively, the number of layers of the various portions of the comfort element is not the same. For example, there can be a single-layered material for the nasal portion and a material having two layers for the frontal portion, or vice versa.

In another alternative solution, the comfort element comprises a first continuous layer common to the various constituent portions, but a second layer specific to each constituent portion. This construction makes it possible to have a unitary comfort element that is easier to handle when not assembled to the frame.

The portions of the comfort element can be assembled to the frame using adhesives, welding, or removable fastening mechanisms.

In another embodiment, the layers and/or portions of the comfort element can be made by bi-injection or overmolding, which makes it possible to obtain a unitary element.

In another alternative embodiment, the comfort element is made by thermoforming from a continuous foam configured to extend around the eyes. The thermoforming operation makes it possible, for example, to vary the thickness of the foam as a function of the support zones. Thus, a zone can be obtained that is thicker in the nasal portion of the comfort element and thinner in the frontal portion of the comfort element. The reduction in thickness, locally, by thermoforming, enables modification of the density of the foam in this zone.

Alternatively, the comfort element can comprise a plurality of distinct thermoformed portions, each configured to be independently affixed to the frame of the mask in relation to the others.

Other foam density values than those described above are within the scope of the invention.

The invention is not limited to the embodiments disclosed. It is possible to combine the disclosed embodiments.

The invention is not limited to the embodiments described above, but extends to all of the embodiments covered by the appended claims.

Further, at least because the invention is disclosed herein in a manner that enables one to make and use it, by virtue of the disclosure of particular exemplary embodiments of the invention, the invention can be practiced in the absence of any additional element or additional structure that is not specifically disclosed herein.

1. An eye protective mask for the practice of outdoor sports, said mask comprising:
an eye protective screen;
a frame supporting the screen;
a comfort element affixed to the frame;
the comfort element comprising:
a nasal portion configured to come into contact with a portion of a user's nose;
a frontal portion configured to come into contact with a portion of the user's forehead;
the nasal portion comprising a nasal portion material and the frontal portion comprises a frontal portion material;
the nasal portion material has different mechanical compression properties from mechanical compression properties of the frontal portion material, such that a maximum extent of compression of the nasal portion material caused by a predetermined maximum compressive force is greater than a maximum extent of compression of the frontal portion material caused by the predetermined maximum compressive force.

2. An eye protective mask according to claim 1, wherein:
at least one of the following comprises a plurality of layers of a variety of different materials:
the nasal portion material;
the frontal portion material.

3. An eye protective mask according to claim 2, wherein:
at least one of the following comprises, successively, from the frame, a first layer of a foam of a first type and a second layer of a foam of a second type, the foam of the first layer having a density greater than a density of the foam of the second layer:
the nasal portion material;
the frontal portion material.

4. An eye protective mask according to claim 3, wherein:
the first layer of the nasal portion material having a thickness between 10% and 30% of an entire thickness of the nasal portion;
the second layer of the nasal portion material having a thickness between 70% and 90% of an entire thickness of the nasal portion;

5. An eye protective mask according to claim 4, wherein:
the thickness of the second layer of the nasal portion is greater than a thickness of the second layer of the frontal portion.

6. An eye protective mask according to claim 5, wherein:
the second layer of the frontal portion and the second layer of the nasal portion are made from an identical type of foam.

7. An eye protective mask according to claim 3, wherein:
the density of the foam of the second layer of the nasal portion is less than the density of the foam of the second layer of the frontal portion.

8. An eye protective mask according to claim 3, wherein:
the foam of the first layer of the nasal portion is a different type than the foam of the first layer of the frontal portion.

9. An eye protective mask according to claim 1, wherein:
a thickness of the nasal portion is greater than a thickness of the frontal portion.

10. An eye protective mask according to claim 9, wherein:
a density of the frontal portion material is greater than a density of the nasal portion material.

11. An eye protective mask according to claim 1, wherein:
a density of the frontal portion material is greater than a density of the nasal portion material.

12. An eye protective mask according to claim 9, wherein:
the nasal portion material and the frontal portion material are made of an identical material.

13. An eye protective mask according to claim 1, wherein:
the nasal portion material is a different type of material than that of the frontal portion.

14. An eye protective mask according to claim 1, wherein:
the comfort element comprises two lateral portions;
the two lateral portions are configured to come into contact with respective temples and respective cheekbones of a the user's face;
each of the two lateral portions connects a respective end of the frontal portion to a respective end of the nasal portion;
each of the lateral portions comprises a lateral portion material having intermediate mechanical compression properties compared to both the nasal portion material and the frontal portion material.

15. An eye protective mask according to claim 1, wherein:
the frontal portion and the nasal portion are two of a plurality of portions of the comfort element;
the plurality of portions of the comfort element are made of various numbers of layers of material.

16. An eye protective mask according to claim 1, wherein:
the frontal portion of the comfort element and the nasal portion of the comfort element are two of at least two distinct elements.

17. An eye protective mask according to claim 16, wherein:
the comfort element comprises four distinct elements comprising the frontal portion, the nasal portion, and two lateral portions.

18. An eye protective mask according to claim 1, wherein:
at least one portion of the comfort element is a thermoformed comfort element portion.

19. An eye protective mask according to claim 1, wherein:
at least one portion of the comfort element is a distinct independent portion configured to be removable from the frame in relation to at least a second portion of the comfort element.

20. An eye protective mask according to claim 1, wherein:
the predetermined maximum compressive force is a horizontally directed force.
